(19) United States
(12) Patent Application Publication
(10) Pub. No.: US 2026/0026844 A1
(43) Pub. Date: Jan. 29, 2026

(54) FRAME OVER NAIL HINDFOOT FIXATION

(71) Applicant: Orthofix S.R.L., Bussolengo (Verona) (IT)

(72) Inventor: William Terrell, Marietta, GA (US)

(21) Appl. No.: 19/282,201

(22) Filed: Jul. 28, 2025

Related U.S. Application Data
(60) Provisional application No. 63/676,707, filed on Jul. 29, 2024.

(51) Int. Cl. A61B 17/66; A61B 17/64; A61B 17/72

(52) U.S. Cl. CPC A61B 17/66; A61B 17/6425; A61B 17/7225

(57) ABSTRACT

A method for applying compression in an ankle includes inserting a proximal portion of a nail through a calcaneus and a talus of a foot of a patient and into a tibia of the patient, securing an external fixation frame to the tibia and the foot, applying compression to the ankle using the external fixation frame, and retaining the compression in the ankle using the nail and one or more screws extending through the nail.

### CROSS-REFERENCED APPLICATIONS

This application claims priority to and benefit of U.S. Provisional Application No. 63/676,707 filed Jul. 29, 2024, entitled “Frame Over Nail Hindfoot Fixation,” which is incorporated by reference herein its entirety.

### TECHNICAL FIELD

The present disclosure relates to systems and methods of ankle arthrodesis. In particular, this disclosure pertains to frame over nail fixation.

### BACKGROUND

Charcot foot is a condition commonly seen in people with peripheral neuropathy, particularly those with diabetes. Charcot foot in diabetics is marked by osteolysis, ligamentous laxity, and architectural collapse of the midfoot and hindfoot, often resulting from peripheral neuropathy and impaired bone healing associated with chronic hyperglycemia. An underlying cause of Charcot foot is nerve damage, which leads to a loss of sensation in the foot. Because the nerves are not functioning properly, individuals may not feel pain or notice injuries such as fractures, sprains, or even minor trauma. As a result, they may continue to walk on the injured foot, causing repeated stress and further damage. Over time, this can lead to the weakening of bones, dislocation of joints, and collapse of the foot's arch, resulting in significant deformity.

Charcot foot is commonly treated with a rigid supportive device, often called a “beam,” to offload pressure from the affected area, reconstitute the longitudinal arch, and stabilize the foot. This method may help prevent further collapse of the foot's structure by distributing weight more evenly and immobilizing joints, allowing the bones to heal and reducing the risk of ulcers or additional deformity. Unfortunately, even after successful beam fixation, many diabetic patients develop late-stage degeneration of the ankle. As a result, additional surgical procedures often follow implantation of a beam such as conversion to a tibiotalocalcaneal (TTC) fusion with an intramedullary hindfoot nail. Although widely adopted, existing systems are associated with several significant shortcomings.

For example, utilizing an intramedullary nail may not accommodate intraoperative variability in patient anatomy or permit dynamic postoperative compression during arthrodesis. Additionally, bone resorption, both preexisting and that which may occur in response to altered biomechanics, may progressively undermine the structural support around the implanted nail. As peri-implant bone stock diminishes, the hardware can migrate or protrude, causing skin ulceration, pain, and heightened risk of infection or amputation, complications that are particularly concerning in the diabetic population.

Accordingly, there remains an unmet need for an intramedullary nail system that accommodates variable anatomy, sustains controlled compression across fusion surfaces, and mitigates hardware prominence.

### SUMMARY

Disclosed herein are systems and methods for performing ankle arthrodesis using a frame over nail treatment.

In an aspect of the present disclosure, a system for ankle compression may include a nail, one or more first screws, and an external fixation frame. The nail may be configured for insertion through a calcaneus and talus of a foot of a patient and into a tibia of the patient. The one or more first screws may be configured for receipt through respective apertures in a distal portion of the nail. The one or more first screws may be configured to be positioned in bone of the calcaneus. The external fixation frame may be configured to be secured at a first end to the tibia and at a second end to the foot with struts connecting a foot portion of the frame to a tibia portion of the frame.

In some examples, the system may further include one or more second screws configured for receipt through respective apertures in a proximal portion of the nail. The one or more second screws may be configured to be positioned in bone of the tibia prior to removal of the external fixation frame.

In an aspect of the present disclosure, a method for applying compression in an ankle may include inserting a proximal portion of a nail through a calcaneus and a talus of a foot of a patient and into a tibia of the patient, securing an external fixation frame to the tibia and the foot, applying compression to the ankle using the external fixation frame, and retaining the compression in the ankle using the nail and one or more screws extending through the nail.

In some examples, the one or more screws may include one or more first screws and the method may further include positioning the one or more first screws in the calcaneus and through one or more apertures in a distal portion of the nail. Positioning the one or more first screws in the calcaneus and through the one or more apertures in the distal portion of the nail may include inserting a longitudinal screw into the calcaneus in a posterior-anterior direction. Positioning the one or more first screws in the calcaneus and through the one or more apertures in the distal portion of the nail may include inserting a transverse screw into the calcaneus in a lateral-medial direction. The one or more first screws may be positioned in the calcaneus and through the one or more apertures in the distal portion of the nail prior to securing the external fixation frame to the tibia and the foot.

In some examples, the one or more screws may include one or more second screws and the method may further include positioning the one or more second screws in the tibia and through one or more apertures in the proximal portion of the nail. The method may include removing the external fixation frame from the tibia and the foot. The one or more second screws may be positioned in the tibia and through the one or more apertures in the proximal portion of the nail after applying the compression to the ankle using the external fixation frame and before removing the external fixation frame.

In some examples, applying the compression to the ankle may include adjusting struts of the external fixation frame to draw a distal ring of the external fixation frame toward a proximal ring of the external fixation frame. Securing the external fixation frame to the tibia and the foot may include securing the proximal ring to the tibia and securing the distal ring to the foot. The proximal ring may be secured to the tibia prior to securing the distal ring to the foot.

In an aspect of the present disclosure, a method for applying compression in an ankle may include inserting a proximal portion of a nail through the ankle of a patient and into a tibia of the patient, after inserting the nail, securing an external fixation frame across the ankle, applying compression to the ankle using the external fixation frame, and after applying the compression to the ankle using the external fixation frame, retaining the compression in the ankle by inserting a screw through the proximal portion of the nail and the tibia.

In some examples, the method may include prior to securing the external fixation frame across the ankle, inserting at least one screw through a distal portion of the nail and a calcaneus of the ankle. The method may include removing the external fixation frame after inserting the screw through the proximal portion of the nail and the tibia.

In some examples, the proximal portion of the nail may be free of screws until the compression has been applied to the ankle. The compression may be applied to the ankle using the external fixation frame over a period of time. The compression may be applied to the ankle using the external fixation frame by adjusting struts connecting a proximal portion of the external fixation frame to a distal portion of the external fixation frame. The struts may be adjusted periodically over the period of time. The period of time may be weeks or months.

In some examples, inserting the proximal portion of the nail through the ankle of the patient and into the tibia of the patient may include inserting the proximal portion of the nail through a calcaneus and a talus.

In some examples, the method may further include extending the tibia after removing the external fixation frame.

### BRIEF DESCRIPTION OF THE DRAWINGS

The accompanying drawing figures incorporated in and forming a part of this specification illustrate several aspects of the disclosure, and together with the description, serve to explain the principles of the disclosure.

FIG. 1 illustrates an example of a method of applying compression in an ankle to facilitate arthrodesis.

FIGS. 2A-2B illustrate an examples of a hindfoot nail that may be used in accordance with the present disclosure.

FIG. 3A illustrates an example of a transverse screw that may be used in accordance with the present disclosure.

FIG. 3B illustrates an example of a longitudinal screw that may be used in accordance with the present disclosure.

FIG. 3C illustrates an examples of an end caps that may be used in accordance with the present disclosure.

FIGS. 4-10 illustrate various steps in a method of applying compression in an ankle to facilitate arthrodesis in accordance with the present disclosure.

FIG. 11 illustrates an example of a hindfoot nail secured at its distal end in accordance with the present disclosure.

FIGS. 12-13 illustrate an example of an external fixation frame that may be used in accordance with the present disclosure.

FIG. 14 illustrates an example of a hindfoot nail secured at its proximal and distal ends in accordance with the present disclosure.

FIG. 15 illustrates an example of lengthening a tibia following an ankle arthrodesis.

The present disclosure is best understood from the following detailed description when read with the accompanying figures.

### DETAILED DESCRIPTION

The present disclosure relates generally to orthopedic fixation devices and, more particularly, to intramedullary compression nail systems configured for use in arthrodesis procedures of the lower extremity. Arthrodesis, or joint fusion, is a surgical technique in which the articular surfaces of two or more bones are permanently joined to alleviate pain, correct deformity, and restore stability that has been compromised by arthritis, trauma, infection, disease, or other pathology. When the ankle joint is implicated, fusion commonly involves the tibia, talus, and, in certain clinical scenarios, the calcaneus, thereby requiring hardware capable of spanning and compressing multiple osseous structures while maintaining proper alignment throughout the postoperative course.

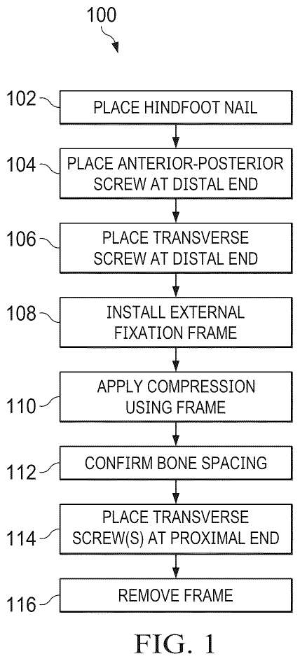

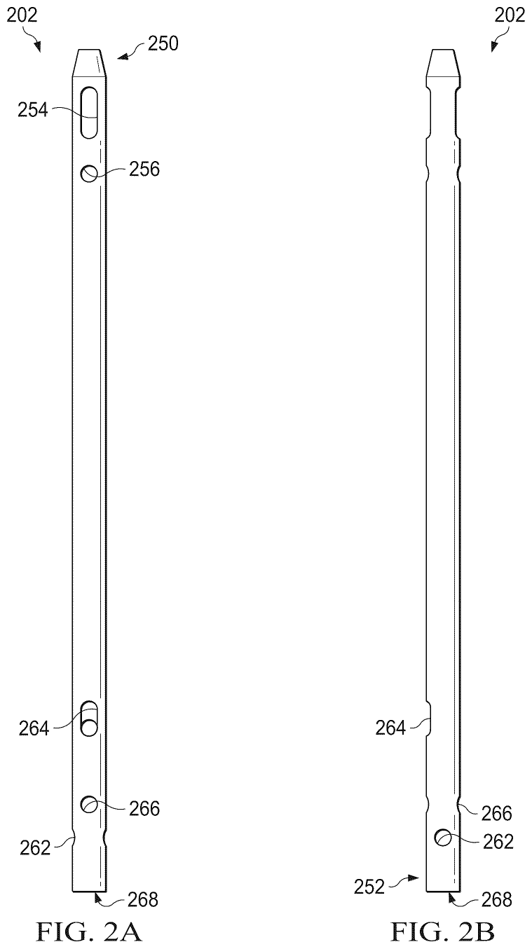

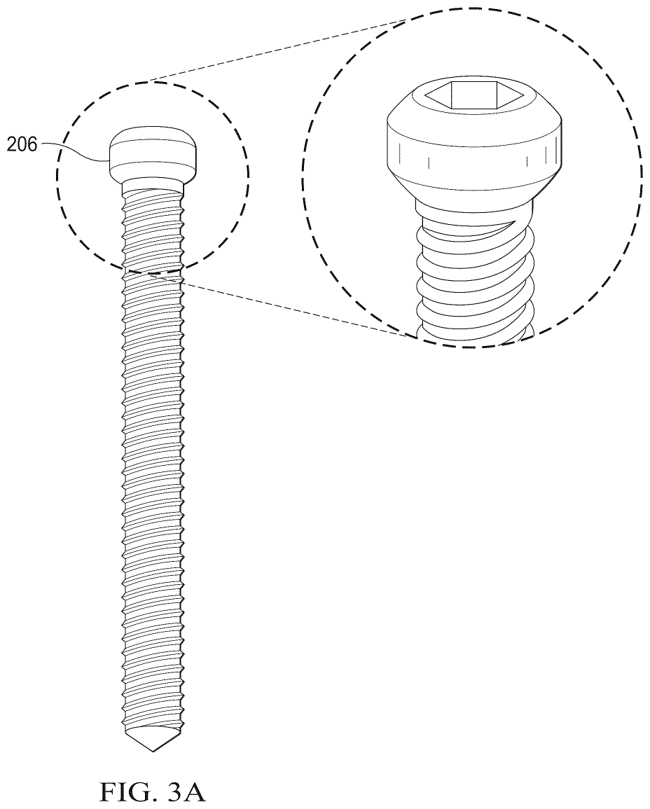

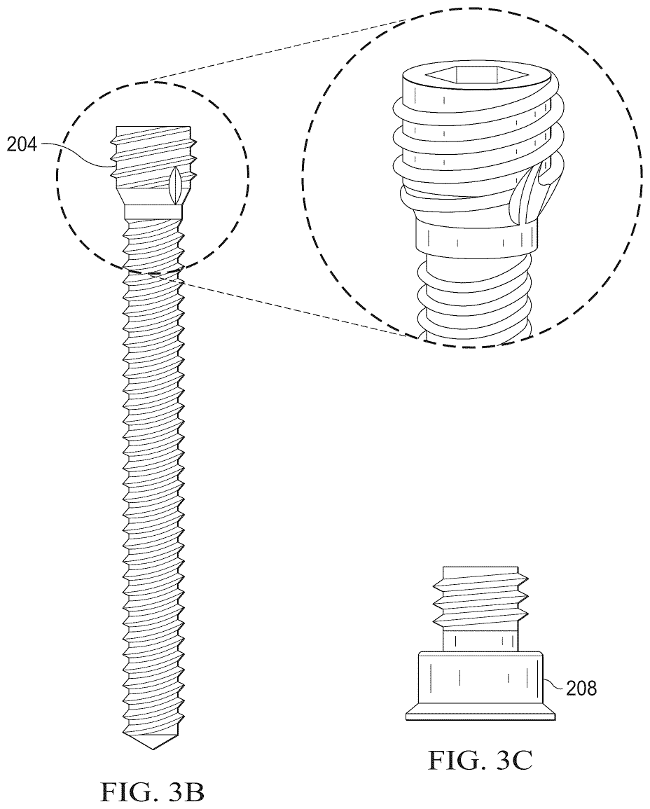

FIG. 1 illustrates an example of a method 100 of applying compression in an ankle to facilitate arthrodesis using a frame-over-nail system. This method may be particularly suited for patients with a Charcot foot diagnosis and may improve compression across the ankle during arthrodesis as compared to conventional hindfoot nail techniques. At process 102, a hindfoot nail is implanted through the ankle. An example of a hindfoot nail 202 is illustrated in FIGS. 2A-2B. The illustrated nail is only one example and it should be appreciated that any suitable diameter and/or length of nail may be utilized. Additionally, while the illustrated nail is substantially straight, a curved nail may also be utilized in suitable circumstances as needed to adapt the procedure to the patient's anatomy. The nail 202 has a proximal end 250 and a distal end 252. A plurality of apertures in the form of slot 254, bore 256, bore 262, slot 264, and bore 266 extend radially through the nail 202. The arrangement of apertures shown in FIGS. 2A-2B is exemplary only and any suitable arrangement of apertures may be used. These apertures are configured to receive corresponding screws, examples of which are shown in FIGS. 3A-3B. Screw 206 is threaded along the shaft with an unthreaded head and is typically used as a transverse screw, placed in a lateral-medial direction into the nail 202 and either the calcaneus or the tibia. Screw 204 is threaded along the shaft and the head and is typically used as a longitudinal screw, placed in a posterior-anterior direction into the nail 202 and the calcaneus. It should be appreciated that screws 204, 206 are exemplary only and any suitable screws may be used which facilitate anchoring of the nail 202 in bone. An opening 268 at the distal end 252 of nail 202 is configured to receive an end cap, an example of which is shown in FIG. 3C. The end cap 208 is configured to lock in place one or more of the screws 204, 206 at the distal end of the nail 202. In some examples, the end cap 208 drives an insert configured to slide longitudinally inside a hollow core of the nail 202 into a respective screw to lock the screw in place.

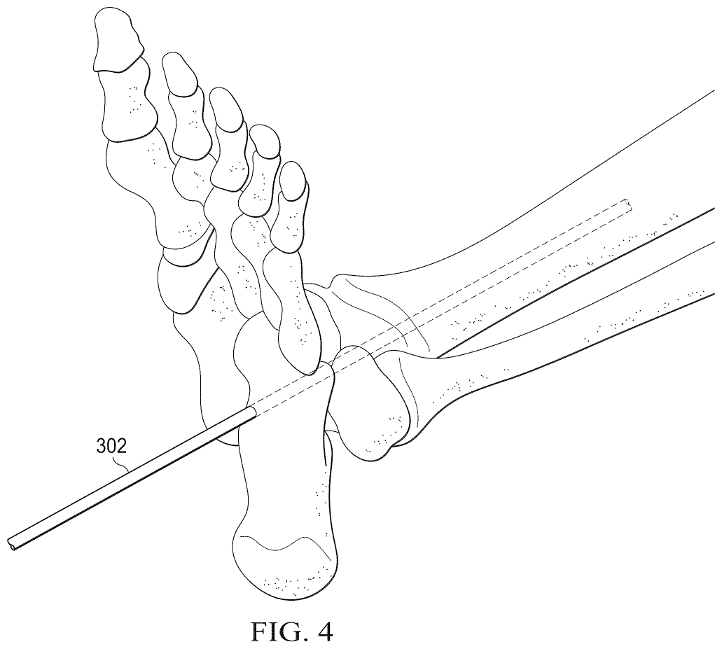

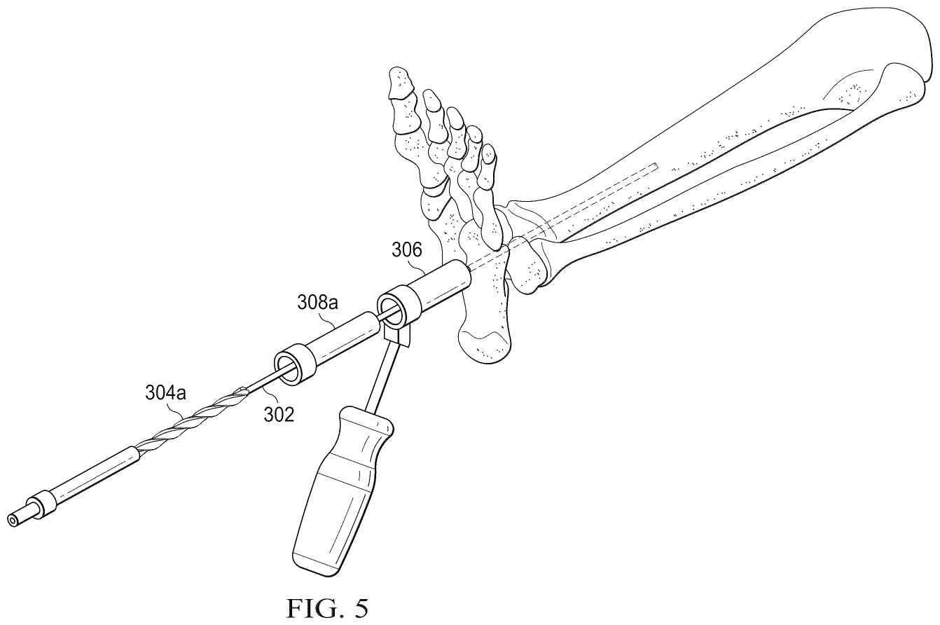

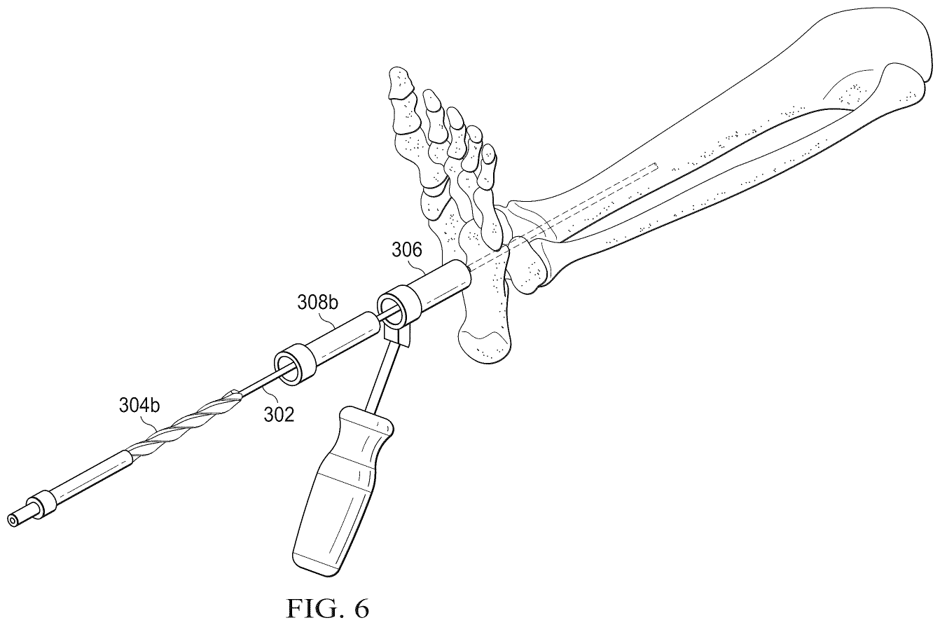

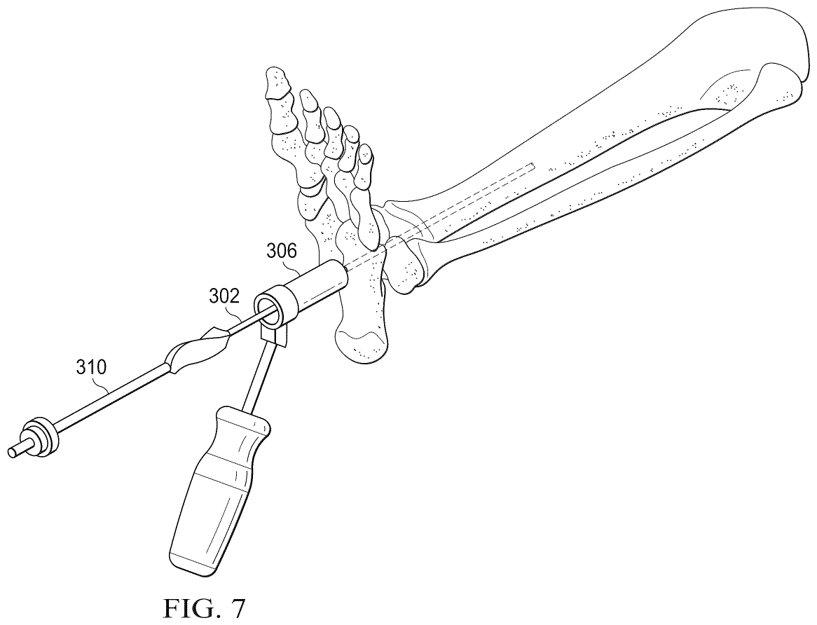

Placement of the nail 202 in process 102 of method 100 may include the steps illustrated in FIGS. 4-8. A guidewire is placed through the calcaneus, the talus, and into the tibia of the patient along a planned path for insertion of instrumentation. A guide 306 may be placed over the guidewire 302. The guide 306 is configured to orient and stabilize various tools used in forming a bore through the patient's bone to receive the nail 202. The bore may be formed by sequential use of drill bits of increasing diameter. As shown in FIG. 5, a first stabilizer insert 308a is placed in the guide 306. The stabilizer insert 308a has a central longitudinal lumen sized to receive a first drill bit 304a to begin enlarging the bore around the guidewire 302 through the calcaneus and talus and into the tibia. As shown in FIG. 6, a second stabilizer insert 308b is placed in the guide 306. The stabilizer insert 308b has a central longitudinal lumen sized to receive a second drill bit 304b to further enlarge the bore around the guidewire 302 through the calcaneus and talus and into the tibia. Any suitable number and size of drill bits may be used to serially enlarge the bore until it properly sized to receive the nail 202. As shown in FIG. 7, a reamer 310 may be driven through the calcaneus, the talus, and the tibia to establish the final diameter of the bore and/or prepare the surface of the bore for receipt of the nail 202.

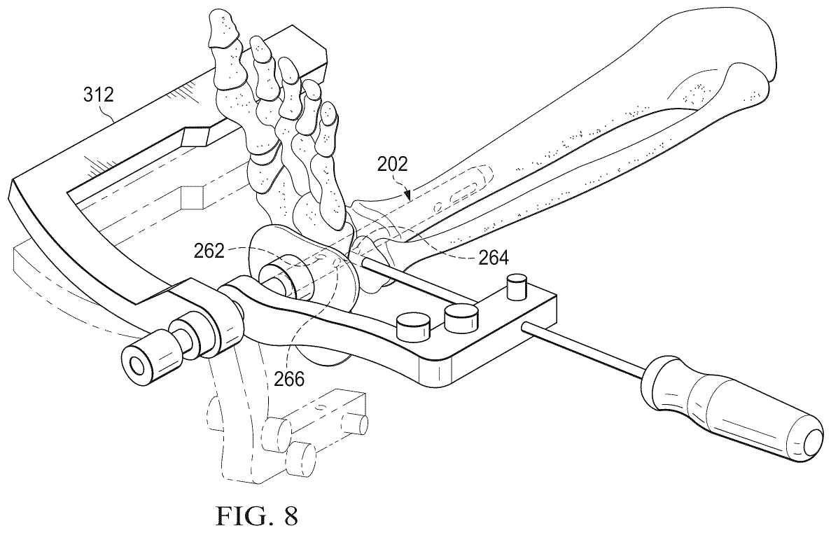

As shown at FIG. 8, a targeting assembly (or jig) 312 holding the nail 202 may be used to insert the nail 202 through the calcaneus and talus and into the tibia. Further information regarding a targeting assembly may be found in U.S. Pat. No. 9,241,744, entitled “TARGETING ASSEMBLY FOR A COMPRESSION NAIL SYSTEM,” which is hereby incorporated by reference in its entirety.

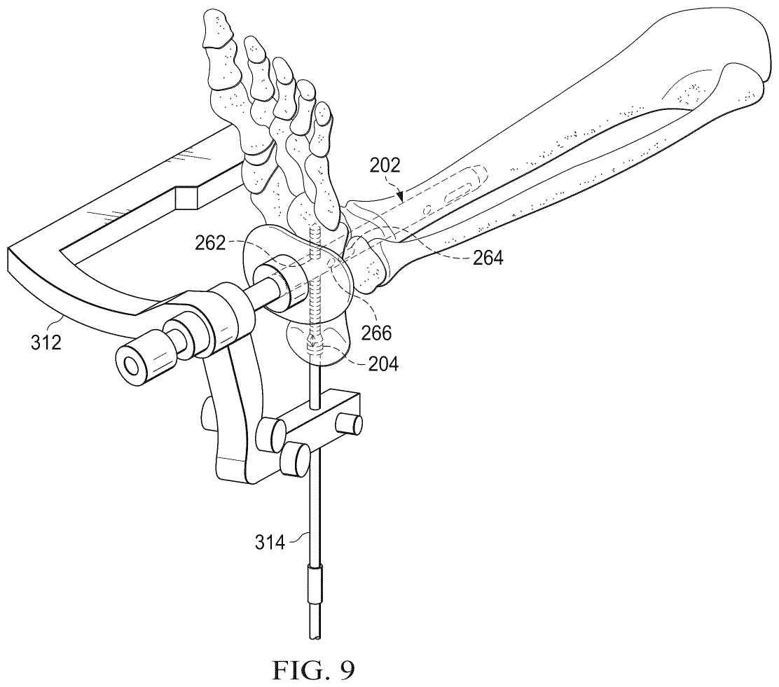

At process 104, a longitudinal screw 204 is placed into the calcaneus in a posterior-anterior direction. This screw 204 may be aligned with and extended through the bore 262 in the distal end 252 of the nail 202 with a screwdriver 314 using the jig 312 as shown in FIG. 9.

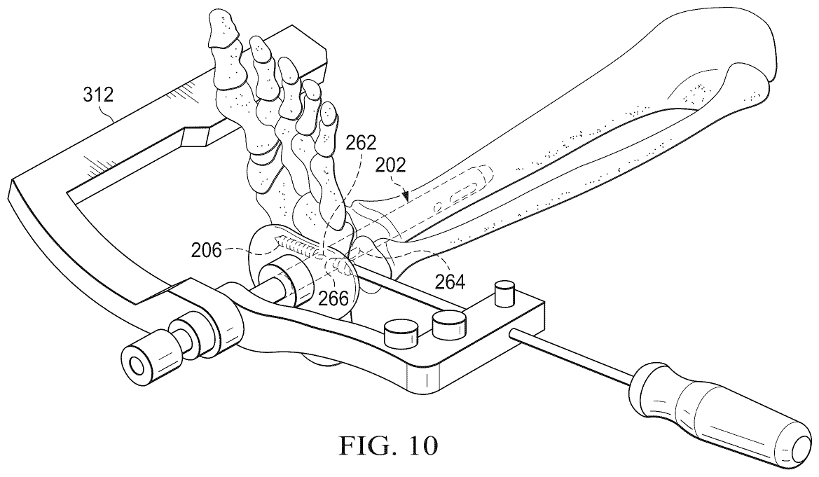

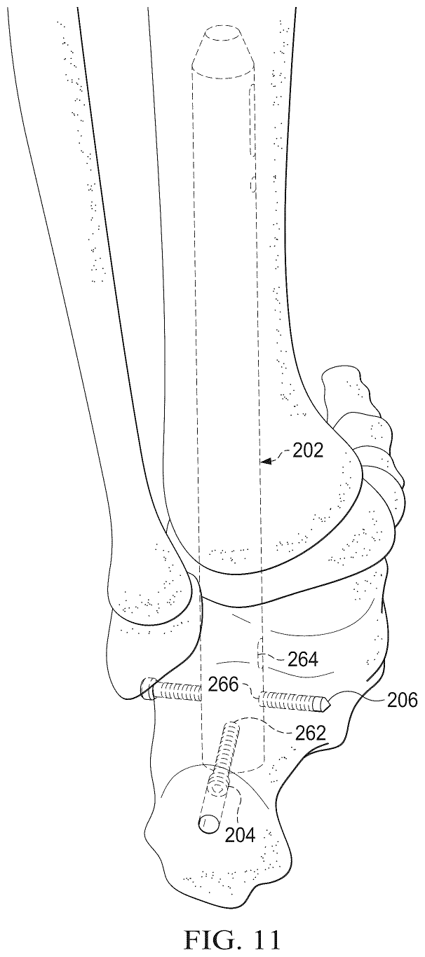

At process 106, a transverse screw 206 is placed into the calcaneus in a lateral-medial direction. This screw 206 may be aligned with and extended through the bore 266 in the distal end 252 of the nail 202 using the jig 312 as shown in FIG. 10. Alternatively, the screw 206 may be aligned with and extended through the slot 264 in the distal end 252 of the nail 202 using the jig 312. In some examples, a plurality of transverse screws may be placed into the calcaneus and extended through the nail 202. For example, a first screw may be placed through slot 264 and a second screw may be placed through bore 266. It should be appreciated that process 106 including placement of transverse screws may alternatively be performed prior to process 104 including placement of a longitudinal screw. FIG. 11 illustrates nail 202 placed within the bore formed in the calcaneus, talus, and tibia with a longitudinal screw 204 and a transverse screw 206 installed to secure the nail 202 with respect to the calcaneus.

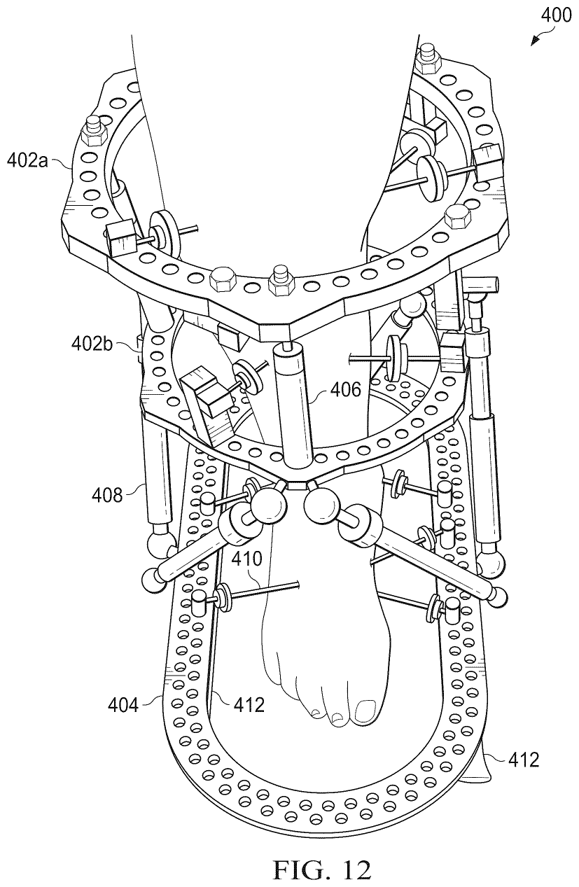

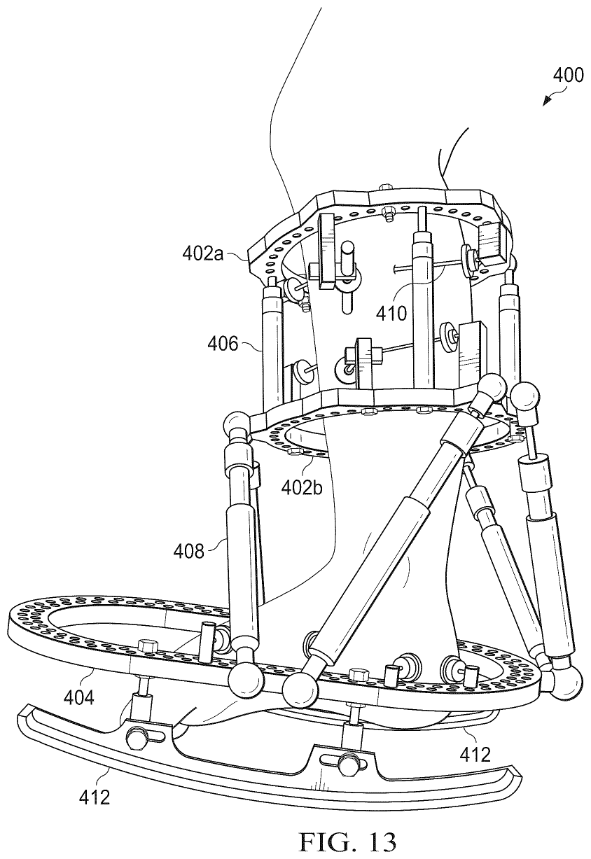

At process 108, an external fixation frame 400 is installed as shown in FIGS. 12-13. The external fixation frame 400 includes a proximal assembly, including two proximal fixation rings 402a, 402b, and a distal assembly including distal fixation ring 404. The proximal fixation ring 402a is connected to proximal fixation ring 402b with a plurality of upper struts 406. The proximal assembly is secured to the patient's tibia with a plurality of pins (or K-wires) 410 extending through the tibia with end portions secured to the proximal fixation rings 402a, 402b. In the illustrated example, each pin 410 extends from one attachment point on a respective fixation ring to an opposing attachment point, passing through the tibia. Alternatively or additionally, half pins may be used. The proximal assembly is connected to the distal assembly by a plurality of lower struts 408 secured at one end to the proximal fixation ring 402b and at the other end to the distal fixation ring 404. The distal assembly is secured to the patient's foot with a plurality of pins 410 extending through the bones of the foot with end portions secured to the distal fixation ring 404. Walking skates 412 extend from the bottom of the distal fixation ring 404 to permit the patient to walk after the external fixation frame 400 is installed. Typically, the proximal assembly will be installed first followed by the distal assembly. It should be appreciated that the illustrated external fixation frame 400 is exemplary only and any suitable external fixation frame may be used. In some examples, on a single proximal fixation ring may be used instead of two proximal fixation rings 402a, 402b connected by upper struts 406. Additional details and examples of an external fixation frame may be found in U.S. Pat. No. 9,456,849, entitled “FAST ADJUST EXTERNAL FIXATION CONNECTION ROD,” U.S. Pat. No. 9,681,892, entitled “EXTERNAL FIXATOR STRUT,” U.S. Pat. No. 9,808,289, entitled, “EXTERNAL FIXATOR RING,” U.S. Pat. No. 9,959,683, entitled “METHOD OF DETERMINING THE POSITION OF AN OBJECT USING PROJECTIONS OF MARKERS OR STRUTS,” and U.S. Pat. No. 10,743,917, entitled “WALKING SKATES SYSTEM REMOVABLY COUPLED TO AN EXTERNAL RING FIXATION SYSTEM,” all of which are hereby incorporated by reference in their entirety. One example of an external fixation frame that may be used in accordance with the present disclosure is the TL-HEX® Truclok Hexapod System available from Orthofix Medical Inc.

At process 110, the external fixation frame 400 is used to apply compression across the ankle. In particular, because the proximal assembly is attached to the tibia and the distal assembly is attached to the foot, the lower struts 408 may be adjusted to shorten their length and, in turn, shorten the distance between the proximal fixation ring 402b and the distal fixation ring 404. This, in turn, drives the calcaneus into the talus and talus into the tibia. Because the distal end 252 of the nail 402 is secured to the calcaneus by screws 204, 206 but the proximal end 250 of the nail 202 is not yet secured to the tibia, the proximal end 250 of the nail 202 is driven further into the tibia in the cephalic direction as the ankle is compressed by the external fixation frame 400. Advantageously, because the nail 202 is implanted prior to applying compression using the external fixation frame 400, the nail 202 serves to maintain the calcancus, talus, and tibia in alignment and prevents these bones from sliding out of alignment with respect to one another in response to the compressive forces applied by the external fixation frame 400.

Although, the compression may be applied statically by the external fixation frame 400, it is primarily contemplated that the compression will be applied dynamically by periodically adjusting the external fixation frame 400 over a period of time. For example, in some instances, the external fixation frame 400 may remain installed on the patient for an extended period of time such as two weeks, a month, two months, or longer. Generally, it is expected that an external fixation frame 400 will be installed on the patient for about eight weeks. During this period of time, the lower struts 408 may be periodically adjusted to further shorten the distance between the proximal fixation ring 402b and the distal fixation ring 404 as the bones in the ankle adjust to the compression. If the bones move with respect to one another during application of compression, the magnitude of compression being applied by the external fixation frame 400 may be reduced. Accordingly, the lower struts 408 may be adjusted to increase the compression back to the desired magnitude. The dynamic application of compression facilitated by the external fixation frame 400 may improve fusion between the calcaneus, talus, and tibia as compared to static compression applied by conventional use of a hindfoot nail and screws without an external fixation frame. That is, conventional use of a hindfoot nail with proximal screws and distal screws installed at the time of implantation of the nail.

At process 112, the spacing between the calcaneus, talus, and tibia may be confirmed. This may be performed immediately after installing the external fixation frame 400 and applying compression at processes 108 and 110, at the end of the period of time during which the external fixation frame 400 is installed on the patient, and/or at any interval of time therebetween. Process 112 may include obtaining one or more radiographic images and evaluating the images to ensure the bones are properly seated against one another. If spacing between the bones is present, the bones will not properly fuse. Accordingly, if spacing is observed, the external fixation frame 400 may be adjusted to drive the bones into engagement. It should be appreciated that processes 110 and 112 may be repeated any suitable number of times throughout application of compression using the external fixation frame 400 over the period of time during which the external fixation frame is installed on the patient.

At process 114, once it has been confirmed that the bones are well-seated without spacing therebetween after a clinically significant period of time to permit arthrodesis, one or more transverse screws 206 may be placed at the proximal end 250 of the nail 202. For example, one or more screws 206 may be placed into the tibia in a medial-lateral direction with the screw(s) aligned with and extending through the slot 254 and/or the bore 256. A “perfect circles” imaging technique may be used to aid in placement these tibial screws through their respective apertures.

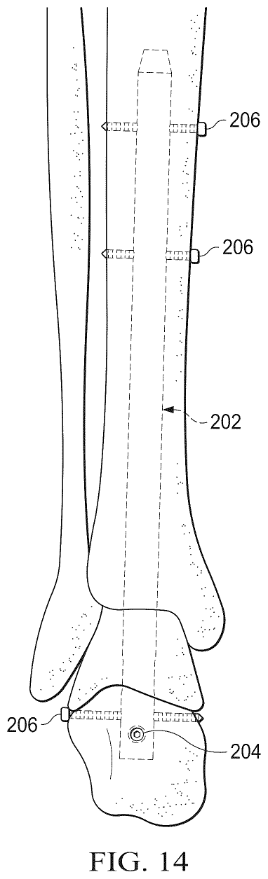

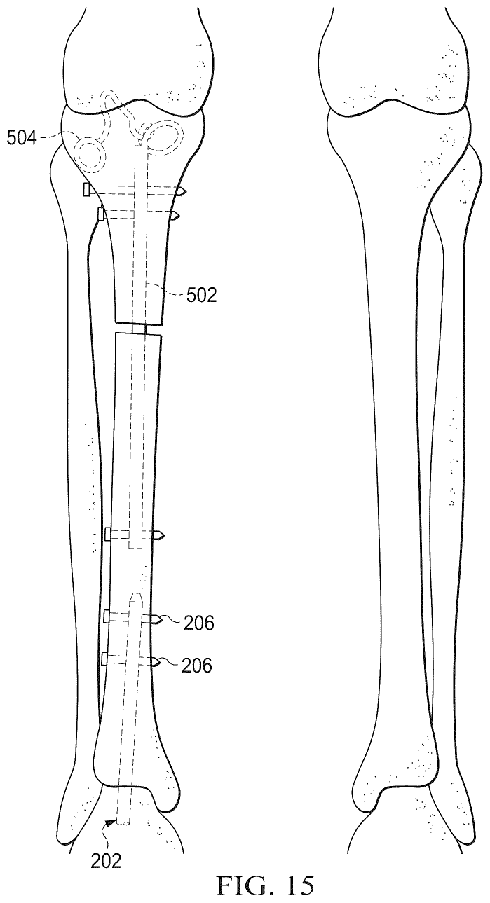

With the distal screws placed in the calcaneus prior to application of compression using the external fixation frame 400 and with the proximal screws placed in the tibia while the external fixation frame is still applying compression across the ankle, the nail 202 retains the ankle in compression. Accordingly, after the proximal screws have been placed, at process 116, the external fixation frame 400 may be removed and the ankle is retained in compression. FIG. 14 illustrates an example of a hindfoot nail 202 secured at its distal end to the calcaneus with two screws and secured at its proximal end to the tibia with two screws 206.

Intra-operative fluoroscopic imaging may be used during various steps of the method 100 to confirm positioning and orientation of the guidewire, hindfoot nail 202, screws 204, 206, and/or pins 410.

It will be appreciated that, advantageously, a frame over nail system and method in accordance with the present disclosure dynamically applies compression to the ankle, may be stronger than conventional hindfoot nail systems, and may be used to correct minor deformities and revise suboptimal procedures. Application of compression to the ankle during the initial recovery period of approximately two months using the external fixation system, rather than the nail, may reduce bone resorption. Further, the use of an external fixation frame with walking skates permits the patient to bear weight earlier in the recovery process than with conventional techniques using only a hindfoot nail. Additionally, the external fixation frame may be used to make rotational adjustment to align the patient's second toe with the patella to obtain optimal alignment during recovery.

Following the frame over nail procedure disclosed herein, the treated leg of the patient may be shortened due to the compression applied across the ankle. Accordingly, in accordance with the present disclosure, a limb lengthening procedure may be performed. One example of a system for such use is the Fitbone™ intramedullary limb-lengthening system available from Orthofix Medical Inc. FIG. 15 illustrates an example of lengthening a tibia following an ankle arthrodesis. A segment of bone may be removed from the tibia at the proximal end. A nail 502 may be placed into the intramedullary canal and secured at its proximal and distal ends with screws. A bone growth stimulation implant may be attached to the nail to stimulate bone growth across the gap formed in the tibia by removal of the bone segment.

While various embodiments in accordance with the principles disclosed herein have been described above, it should be understood that they have been presented by way of example only, and are not limiting. Thus, the breadth and scope of the invention(s) should not be limited by any of the above-described exemplary embodiments, but should be defined only in accordance with the claims and their equivalents issuing from this disclosure. Furthermore, the above advantages and features are provided in described embodiments, but shall not limit the application of such issued claims to processes and structures accomplishing any or all of the above advantages.

It will be understood that the principal features of this disclosure can be employed in various embodiments without departing from the scope of the disclosure. Those skilled in the art will recognize, or be able to ascertain using no more than routine experimentation, numerous equivalents to the specific procedures described herein. Such equivalents are considered to be within the scope of this disclosure and are covered by the claims.

The foregoing outlines features of several embodiments so that those skilled in the art may better understand the aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions, and alterations herein without departing from the spirit and scope of the present disclosure.

What is claimed is:

1. A method for applying compression in an ankle, comprising:
inserting a proximal portion of a nail through a calcaneus and a talus of a foot of a patient and into a tibia of the patient;
securing an external fixation frame to the tibia and the foot;
applying compression to the ankle using the external fixation frame; and
retaining the compression in the ankle using the nail and one or more screws extending through the nail.

2. The method of claim 1, wherein the one or more screws comprise one or more first screws and the method further comprises positioning the one or more first screws in the calcaneus and through one or more apertures in a distal portion of the nail.

3. The method of claim 2, wherein positioning the one or more first screws in the calcaneus and through the one or more apertures in the distal portion of the nail comprises inserting a longitudinal screw into the calcaneus in a posterior-anterior direction.

4. The method of claim 3, wherein positioning the one or more first screws in the calcaneus and through the one or more apertures in the distal portion of the nail comprises inserting a transverse screw into the calcaneus in a lateral-medial direction.

5. The method of claim 2, wherein the one or more first screws are positioned in the calcaneus and through the one or more apertures in the distal portion of the nail prior to securing the external fixation frame to the tibia and the foot.

6. The method of claim 1, wherein the one or more screws comprise one or more second screws and the method further comprises positioning the one or more second screws in the tibia and through one or more apertures in the proximal portion of the nail.

7. The method of claim 6, further comprising removing the external fixation frame from the tibia and the foot, wherein the one or more second screws are positioned in the tibia and through the one or more apertures in the proximal portion of the nail after applying the compression to the ankle using the external fixation frame and before removing the external fixation frame.

8. The method of claim 1, wherein applying the compression to the ankle comprises adjusting struts of the external fixation frame to draw a distal ring of the external fixation frame toward a proximal ring of the external fixation frame.

9. The method of claim 8, wherein securing the external fixation frame to the tibia and the foot comprises securing the proximal ring to the tibia and securing the distal ring to the foot.

10. The method of claim 9, wherein the proximal ring is secured to the tibia prior to securing the distal ring to the foot.

11. A method for applying compression in an ankle, comprising:
inserting a proximal portion of a nail through the ankle of a patient and into a tibia of the patient;
after inserting the nail, securing an external fixation frame across the ankle;
applying compression to the ankle using the external fixation frame; and
after applying the compression to the ankle using the external fixation frame, retaining the compression in the ankle by inserting a screw through the proximal portion of the nail and the tibia.

12. The method of claim 11, further comprising:
prior to securing the external fixation frame across the ankle, inserting at least one screw through a distal portion of the nail and a calcaneus of the ankle.

13. The method of claim 12, further comprising:
removing the external fixation frame after inserting the screw through the proximal portion of the nail and the tibia.

14. The method of claim 11, wherein the proximal portion of the nail is free of screws until the compression has been applied to the ankle.

15. The method of claim 11, wherein the compression is applied to the ankle using the external fixation frame over a period of time.

16. The method of claim 15, wherein the compression is applied to the ankle using the external fixation frame by adjusting struts connecting a proximal portion of the external fixation frame to a distal portion of the external fixation frame.

17. The method of claim 16, wherein the struts are adjusted periodically over the period of time.

18. The method of claim 17, wherein the period of time comprises at least one month.

19. The method of claim 11, wherein inserting the proximal portion of the nail through the ankle of the patient and into the tibia of the patient comprises inserting the proximal portion of the nail through a calcaneus and a talus.

20. The method of claim 11, further comprising:
extending the tibia after removing the external fixation frame.
